(19) United States
(12) Patent Application Publication
(10) Pub. No.: US 2025/0176818 A1
(43) Pub. Date: Jun. 5, 2025

(54) MEDICAL APPARATUS

(71) Applicant: LUTZ FREITAG, Hemer (DE)

(72) Inventor: LUTZ FREITAG, Hemer (DE)

(21) Appl. No.: 18/841,616

(22) PCT Filed: Feb. 27, 2023
(86) PCT No.: PCT/DE2023/100152

§ 371 Date: Aug. 26, 2024

(30) Foreign Application Priority Data
Feb. 28, 2022 (DE) 10 2022 104 714.8

(51) Int. Cl. A61B 1/267; A61B 1/00

(52) U.S. Cl. CPC A61B 1/2676; A61B 1/00128

(57) ABSTRACT

A medical apparatus includes a measuring adapter designed for detachable connection to different respiratory gas pathway devices which have ventilation parameters. A first sensor is arranged on the measuring adapter and designed to measure a measured variable of the different respiratory gas pathway devices. An evaluation unit is designed to assign correction factors to the ventilation parameters based on measured values of the first sensor.

The invention relates to a medical apparatus according to the features of patent claim 1.

Endoscopes are used for minimally invasive surgical procedures on humans and animals as well as in technology for visual inspection of cavities that are difficult to access. Depending on the intended use, endoscope tubes with different diameters find application. In bronchoscopy, strong bronchoscopes made of stainless steel with wall thicknesses of 0.6 mm find oftentimes application. Diameters in the range of 10 mm with lengths of approx. 400 mm are customary. Tube diameters of 10-16 mm are required to place the frequently used silicone stents in correspondence to the width of a normal larynx. Handling thicker devices for stent placement requires practice and experience. During endoscopy, it is not possible to change from a smaller diameter to a larger diameter or vice versa without great effort. It is normally necessary to completely exchange the endoscope tube, including the endoscope head attached to it, and to reattach all the connections arranged thereto, In bronchoscopy, the connections include, in particular, connections for ventilating the lungs and also connections for measuring the pressure conditions within the endoscope.

DE 10 2020 110 840 A1 discloses an endoscope head that enables use of different endoscope tubes in a simple manner and to realize connections with the endoscope head as easily as possible. At least one measuring channel is arranged in the endoscope head. The measuring channel has an inner connection opening, which is connected to a longitudinal channel in the endoscope head, and an outer connection opening, which is arranged on the outside of the endoscope head. The outer connection opening is connectable to a measuring adapter, which in turn can be detachably coupled to the endoscope head. The measuring adapter serves as a quick-action coupling to a measuring instrument, which is connected to the measuring adapter in a gas-conducting manner. If there are several measuring channels in the endoscope head, the measuring adapter can be connected to the endoscope head via a single plug-in motion without having to connect each measuring channel individually. The measuring adapter is preferably configured as a U-shaped clamp.

The use of such a measuring adapter offers considerable advantages. In the case of bronchoscopy, however, the fact that changing the diameter of the endoscope tube results in different boundary conditions for ventilation complicates matters. Ventilation is usually provided by a turbine and/or a jet ventilator. The traction volume depends largely on the diameter of the endoscope tubes. As diameters become smaller and the cross-sectional area of the endoscope tube decreases by a square, the differences become more and more significant, i.e. the traction volume decreases very sharply. The operator is required to set the correct ventilation volume and to ensure the desired ventilation depending on the design of the endoscope head or the used endoscope tube.

The invention is based on the object to provide a medical apparatus that facilitates the setting of ventilation parameters.

This object is attained in a medical apparatus according to the features of patent claim 1.

The subclaims relate to advantageous refinements of the invention.

The medical apparatus includes a measuring adapter which is designed and configured to be detachably connected to different respiratory gas pathway devices. A respiratory gas pathway device within the meaning of the invention is in particular an endoscope head, an endoscope tube, an intubation tube, but also a breathing hose, a breathing tube of a spirometer and also a bite block which has a respiratory gas guide. In terms of its basic functionality, the measuring adapter can be designed as in DE10 2020 110 840 A1, I.e. it can be easily coupled mechanically without having to connect measuring lines individually when changing the endoscope.

The term respiratory gas pathway devices includes devices that are partially or completely arranged inside or outside a patient's respiratory tract. In particular, the term includes devices that lead from a respiratory gas source to a patient's breathing opening. An endoscope head and a spirometer or breathing tube of a spirometer that can be coupled to the endoscope head as well as a breathing gas hose are examples of respiratory gas pathway devices that are arranged completely outside a patient's respiratory tract. An endoscope tube or intubation tube is partially located in the patient's respiratory tract during use. A mouthpiece of a breathing tube of a spirometer is also partially located in the upper respiratory tract of a patient during use, while the respiratory gas enters the breathing tube of the spirometer from an external breathing air source. Even when a respiratory gas inlet and also a respiratory gas outlet of a certain respiratory gas pathway device, like e.g. an external spirometer, which is placed anteriorly of or connected to an endoscope head, are completely outside the patient's natural respiratory tract, i.e. in particular outside the upper respiratory tract (nasal cavity, oral cavity, pharynx), respiratory gas pathway devices are involved within the meaning of the invention.

When using the measuring adapter with a breathing tube of a spirometer, the term “ventilation parameter” relates to any parameters of the spirometer that influence ventilation.

According to the invention, the measuring adapter includes in addition at least one sensor. The sensor is intended to determine properties which are associated with different respiratory gas pathway devices, in particular certain designs of the endoscope head and/or the endoscope tube and/or the intubation tube and which have an impact on ventilation. In particular, conclusions are to be drawn about the cross-sectional area of the endoscope tube or the intubation tube or other respiratory gas pathway devices in the respiratory gas pathway through which breathing air can flow. When determining a smaller cross-sectional area than a previously defined reference value, at least one ventilation parameter is to be adjusted. The degree of adjustment is expressed as a correction factor. In an evaluation unit, previously stored correction factors for the at least one ventilation parameter are assigned to the measurement data of the sensor. The correction factors can be displayed to enable manual adjustment of a respiratory gas source. The correction factors can also be used for automated adjustment of the ventilation parameters.

The sensor is used to determine a measured variable, e.g. of the endoscope head or the endoscope tube. A measured variable may also be a marking or coding. She measured coding makes it possible to assign the design to a correction factor.

According to a first embodiment, the sensor converts information from an information carrier on the respiratory gas pathway device, in particular on the endoscope head, endoscope tube or intubation tube, into a signal, in particular an electrical signal. The correction factor is assigned to this signal. The correction factor is displayed.

The information carrier on the respiratory gas pathway device, in particular on the endoscope tube, endoscope head or intubation tube, contains information about the type of respiratory gas pathway device, in particular the endoscope head or the type of endoscope tube or the type of intubation tube. The term intubation tube refers to tubes that are inserted into the mouth or nose to establish a safe respiratory tract. With the aid of intubation tubes unconscious, sedated or anaesthetized patients can be ventilated. The term includes both endotracheal tubes and laryngeal tubes.

The information on the information carrier is preferably an optical code, in particular a barcode. Information carriers on the respiratory gas pathway device, in particular on the endoscope tube, endoscope head or intubation tube, can have binary (b/w) or multicolored coding. The readout is contactless.

In principle, it is also possible to mechanically read out mechanically scannable information in the form of raised and non-raised regions, such as e.g. discrete dots similar to Braille, roughness of the surface or another tactile information, for example using one or more displacement transducers or mechanical scanners. The invention also includes electromagnetic information carriers, such as e.g. radio-frequency systems (oscillating circuits, RFID tags). Near Field Communication (NFC) in particular is very well suited for the contactless exchange of data by electromagnetic induction using loosely coupled coils over short distances of a few centimeters. Communication is active-passive in particular.

A correlation of measurement data to the correction factors of ventilation parameters of different respiratory gas pathway devices, in particular endoscope heads and endoscope tubes, is stored in a memory unit in order to make it available to the evaluation unit. This may involve one or more correlation functions, particularly in the case of analog sensors. The correlation can also include a discrete assignment. Correction factors can be transferred to the memory unit via an interface in coordination with the used respiratory gas pathway device, in particular endoscope heads, endoscope tubes or intubation tubes.

The ventilation unit and/or the measuring adapter include in particular a display unit to signal which correction factor is required and preferably also which type of respiratory gas pathway device, in particular an endoscope head, endoscope tube or intubation tube, is connected to the measuring adapter.

The ventilation unit is preferably connected to the measuring adapter in a data-transmitting manner, with a control unit of the ventilation unit being designed to automatically adjust the ventilation parameters while taking into account the correction factors. The ventilation parameters are preferably adjusted immediately after the data transfer. The adjustment is realized continuously. Optionally, provision may be made for the adjustment to be acknowledged manually in advance.

The signal or the correction factor can be transmitted from the measuring adapter to a receiver unit. The receiver unit is in turn connected to the ventilation unit, i.e. the respiratory gas source, or is itself a component of the ventilation unit. In particular, the receiver unit can be retrofitted and can be coupled to the ventilation unit via an interface.

The medical apparatus according to the invention relates, with regard to the respiratory gas pathway device, in particular both to endoscope heads in which endoscope tubes of different diameters can be connected and to endoscope heads with unchangeable endoscope tubes. In the case of endoscope heads in which the endoscope tube is interchangeable, the information as to which endoscope head is connected is not sufficient to identify the current diameter of the endoscope tube. In an advantageous refinement of the invention, the measuring adapter can identify endoscope tubes of different diameters. This is realized by means of at least one second sensor. The measured variable is converted into a measurement signal by the at least one second sensor and evaluated. The diameter is determined and, in particular, measured without contact, preferably by optical measurements. Sensors can be used as a component of light barriers. The scattered light from a light source may also be measured, which in turn allows conclusions to be drawn about the diameter of the connected endoscope tube. The measurement can also be carried out using ultrasonic sensors. The invention includes the use of several sensors in order to increase the measurement accuracy or to combine measurement methods with each other or to enable redundant measurement.

When, for example, only two different endoscope tubes can be connected to an endoscope head, it may be sufficient to ascertain which endoscope tube is connected. In this case, it is sufficient for the second sensor to determine any distinguishing feature, e.g. a different light transmission as an identifier of the endoscope tube. The invention therefore includes determining different diameters by means of the second sensor without measuring the diameter directly.

In an advantageous refinement of the invention, the measuring adapter includes at least one sensor which is configured and designed to ascertain the presence of an instrument in the endoscope tube or in the intubation tube without contact. This may involve the same sensor that is used to ascertain the diameter or at least one other sensor.

An inserted instrument has a considerable influence on the flow conditions within the endoscope tube or the intubation tube, wherein an adequate ventilation of the patient must also be ensured via the remaining annular space between the instrument and the wall of the endoscope tube or the intubation tube. The sensor is used to ascertain at least the presence of an instrument in the endoscope tube or in the intubation tube. The endoscope tube or the intubation tube are designed to enable this measurement. For this purpose, the endoscope tube or the intubation tube has at least one region that is permeable to the measured variable of the at least one sensor. In the case of endoscope tubes or intubation tubes made of plastic, in particular of translucent plastic, the entire endoscope tube or the entire intubation tube can be regarded as a permeable region for the measured variable of the at least one sensor. In the case of endoscope tubes made of metal, on the other hand, a correspondingly permeable region is required.

The signal gained by the at least one sensor is assigned to correction factors, or the correction factors are derived from the measured values. The presence and/or the dimension of an instrument in the endoscope tube or in the intubation tube can be taken into account as a correction factor. The dimension of an instrument relates in particular to the diameter of the instrument in the endoscope tube or in the intubation tube. The term “instrument” is representative of any body that is inserted into the channel of the endoscope tube or into the intubation tube and thus has an impact on the cross-sectional ratios in the endoscope tube or in the intubation tube. According to the invention, the aim is to be able to determine the freely flowable annular space within the endoscope tube or in the intubation tube as accurately as possible in order to be able to match the ventilation parameters to the respective situation better and, in particular, automatically in real time.

The region that is permeable for the measured variable is in particular optically permeable, i.e. permeable to light. The term “light” does not include any restriction to a specific wavelength or to visible light. The term light within the meaning of the invention also includes the infrared range. Consequently, infrared sensors can, for example, also be used to ascertain the transmission of the light signal through a permeable region of the endoscope tube or the intubation tube. All sensors of the measuring adapter according to the invention are connected in particular to a signal-processing unit that is integrated into the measuring adapter.

A region that is permeable with regard to the measured variable can be used for ultrasound measurements. The at least one sensor can be an ultrasound probe.

When carrying out an optical measurement, the translucent region can extend in particular over the entire circumference of the endoscope tube or the intubation tube. This makes it possible to arrange the measuring clamp in any radial orientation on the endoscope tube or on the intubation tube when the measuring adapter is not placed directly on the endoscope head.

Depending on the measuring method, it may be necessary to generate the measured variables, e.g. ultrasound or light of a specific wavelength. The use of Hall sensors or magnetic sensors also requires corresponding measured variables. In a refinement, the invention therefore provides arrangement of at least one signal source for generating the measured variable of the at least one sensor on the measuring adapter and/or on the respiratory gas pathway device, in particular on the endoscope head and/or endoscope tube and/or on the intubation tube. The signal source may, for example, involve a light source. For optical measurements in particular, the invention provides for an opposing and in particular diametrical arrangement of a signal source and a sensor, comparable to the principle of a light barrier. Magnets, voice coils, RFID tags, optical markings, changing material compositions, etc. can be detectable features via sensors. The invention is not limited to a specific physical measuring principle. The decisive factor for the choice of measuring method is the ability to assign a sufficiently accurate correction factor to the measured value.

With all possible sensors that can find application, the measured variable is converted into an electrical signal. The sensor signal can be transmitted wirelessly from the measuring adapter to the ventilation unit. It is not necessary for the entire transmission path to be wireless. The ventilation unit can be assigned an external, in particular retrofittable, receiver that serves as a receiving unit. The external receiver is in turn connected to the ventilation unit by cable.

An energy source, in particular a battery or a rechargeable accumulator, can be arranged in the measuring adapter for signal transmission. In particular, the energy source in the measuring adapter can be charged without contact. The measuring adapter includes a transmitter in particular.

The measuring adapter according to the invention fulfills in particular the same basic functions as in DE 10 2020 110 840 A1, i.e. it has at least one measuring channel which corresponds to measuring channels in the endoscope head with regard to position. A fluid-conducting, in particular gas-conducting, connection to external connections of measuring channels in the endoscope head is established via the measuring channels. Information about the pressure conditions inside the endoscope head can be gained via the measuring channels.

The measuring adapter is preferably configured as a U-shaped clamp with a back and two arms that are connected to the back. In this way, the measuring adapter can be detachably coupled to a respiratory gas pathway device, in particular an endoscope head, endoscope tube or intubation tube. It serves as a link and, in particular, as a quick-action coupling between a measuring device, which is connected, for example, to the measuring adapter via a hose and the respiratory gas pathway device, in particular the endoscope head, endoscope tube or intubation tube. The multiple measuring channels in the endoscope head can be connected via a single plug-in motion of the measuring adapter. In this way, it is possible to prepare the endoscope head for use in a simple and uncomplicated manner.

The measuring adapter is preferably held on the respiratory gas pathway device, in particular on the endoscope head, endoscope tube or intubation tube, using a clamping force. At least one of the two arms may hereby be designed resilient for this purpose. In particular, the entire measuring adapter is made of a resilient material, so that both arms can be resilient due to the material properties. The spring force can alternatively or additionally be built up by elastic deformation of the back. The arms are slightly bent apart when they are laterally attached to the endoscope head. Arranged on the respiratory gas pathway device, in particular on the endoscope head and/or on the free ends of the arms are preferably inclined surfaces which slide against each other so that the clamp is bent open. When the chamber is in the final position, the arms spring back and lie clamped and sealed against the respiratory gas pathway device, in particular against the endoscope head. In like manner, the clamp can be attached to other respiratory gas pathway devices, in particular the endoscope tube or the intubation tube. It is considered advantageous when the connection to the respiratory gas pathway device, in particular to the endoscope head, is not only clamped, i.e. by a force fit, but is in addition or as an alternative by a form-fit. A form-fit connection in the direction of attachment or in opposition to the direction of attachment changes the slippage of the measuring adapter with appropriately selected tolerances and thus an incorrect position, incorrect operation or falsification of measured values. Provision is therefore made for at least one arm, and in particular both arms, to have latching projections by which the measuring adapter can be form fittingly coupled to the respiratory gas pathway device, in particular to the endoscope head. A form fit in the direction of attachment may already be achieved by the back of a U-shaped clip coming into contact with a contact portion on the respiratory gas pathway device, in particular the endoscope head. As a result, the attachment depth can be limited. Lateral guides for one or both arms on the arms and/or on the respiratory gas pathway device, in particular on the endoscope head, ensure the position of the measuring adapter in longitudinal direction of the respiratory gas pathway device, in particular the endoscope head. It is considered particularly beneficial when a combination of a form-fit connection for position orientation and force-fit connection for sealing the transition in the area of the outer connection openings of the measuring channels is involved.

Sensors can be integrated using a multi-part design of the measuring adapter, which serves virtually as a carrier or housing for the sensors. The housing carries sensors, includes measuring channels and accommodates microelectronics and optionally an energy store. The housing is therefore a multifunctional component that goes far beyond the function of a pure adapter or a clamp for fixation.

In a particularly advantageous manner, the at least one first sensor is located in the back of the U-shaped clamp. In particular, the lateral arrangement of the measuring adapter is always in the same position on the respiratory gas pathway device, especially on the endoscope head, so as to always ensure that the first sensor is able to also read the information carrier on the respiratory gas pathway device, especially on the endoscope head. A further sensor is preferably arranged in or on the arm. In the case of sensors that require signal sources to generate the measured variable, the corresponding sensor and/or the signal source is preferably located on at least one of the arms. The arms enable the opposing and preferably diametrical arrangement of signal sources and sensors in order to ascertain the diameter or dimension of an instrument in the endoscope tube.

With regard to the sensors, both analog and digital sensors can be considered. Analog signal detection makes it possible to evaluate the scattering of a light signal and to gain additional information as to whether an instrument is located for example in a translucent region of an endoscope tube or an intubation tube. The instrument leads to formation of a shadow that can be measured. Using a correction factor and knowing the diameter of the endoscope tube or intubation tube, it is possible to determine how the jet stream or a turbine of a respiratory gas source for supplying breathing air must be operated in order to be able to ventilate the patient to the desired extent even when the instrument is inserted. When the instrument is removed again, a control unit regulates the ventilation pressure or the ventilation volume, preferably automatically, back to the desired value. For the application according to the invention in combination with translucent endoscope tubes or intubation tubes, analog sensors offer the possibility to gain information both with regard to the diameter of the endoscope tube or intubation tube and with regard to the instrument inserted into the endoscope tube or intubation tube by using one or optionally several light barriers which are positioned in such a way that at least one of the light barriers provides information about the diameter of the endoscope tube or intubation tube and, optionally, another light barrier provides information about the presence of an instrument in the endoscope tube or in the intubation tube. Ideally, only a single additional sensor is required to gain sufficiently accurate measured values for an appropriate correction factor.

The invention is explained hereinafter with reference to exemplified embodiments shown in purely schematic drawings.

It is shown in:

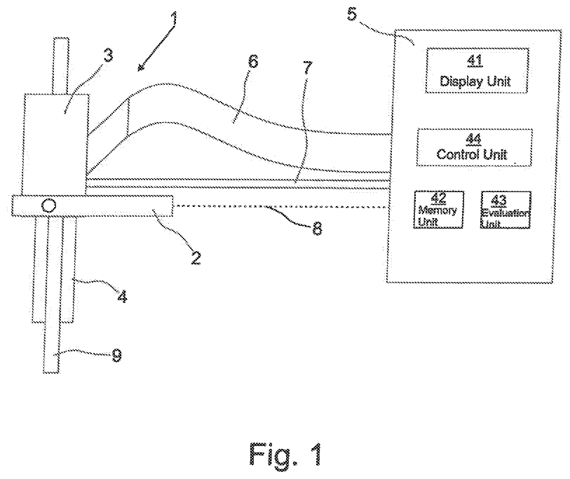

FIG. 1 a schematic view of a medical apparatus;

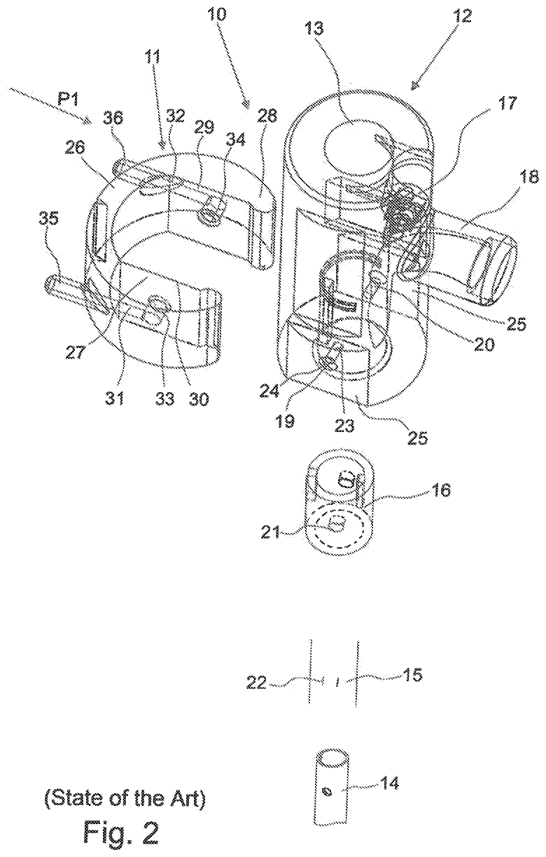

FIG. 2 an endoscope head according to the state of the art;

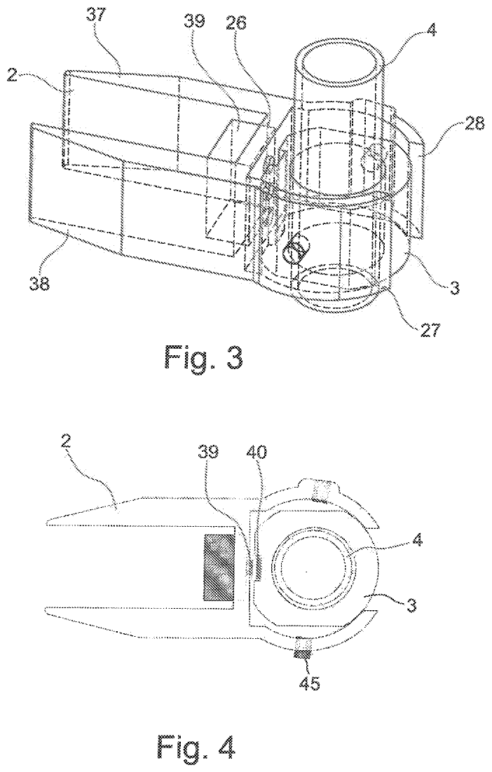

FIG. 3 a perspective view of a first embodiment of the invention;

FIG. 4 a cross-section of the embodiment of FIG. 3;

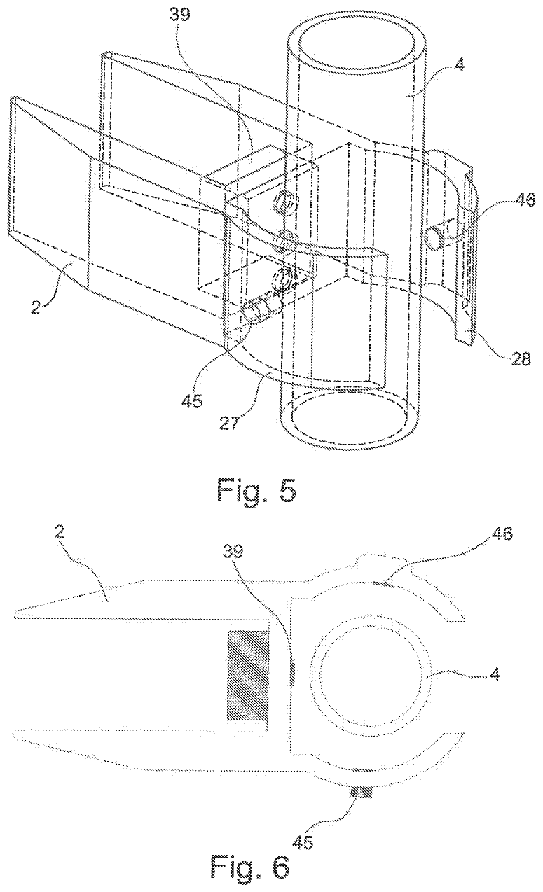

FIG. 5 a perspective view of a further embodiment;

FIG. 6 a cross-section of the embodiment of FIG. 5;

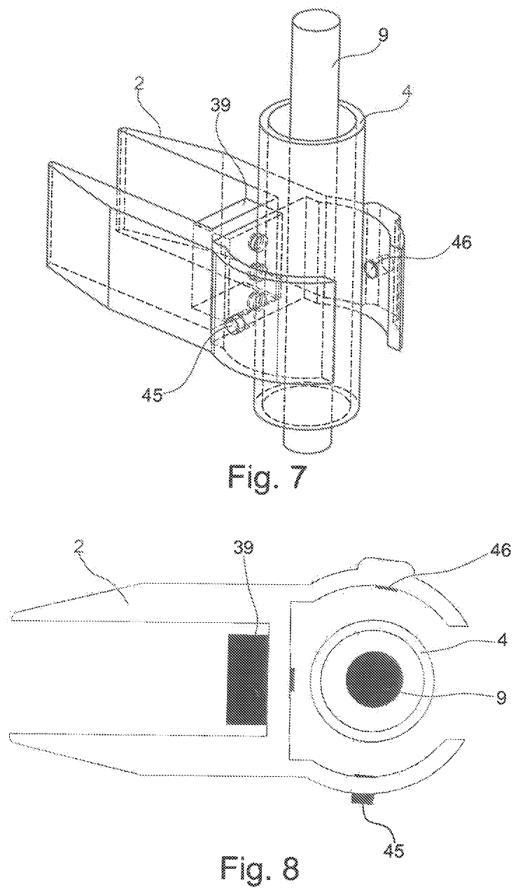

FIG. 7 the embodiment of FIG. 5 in a further application;

FIG. 8 a cross-section of the embodiment of FIG. 7;

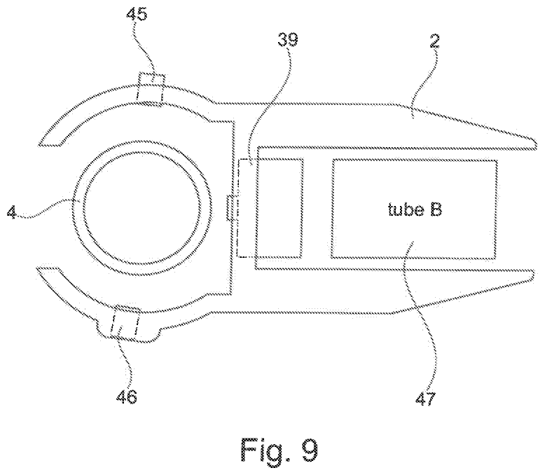

FIG. 9 a cross-section of a further embodiment of a measuring adapter;

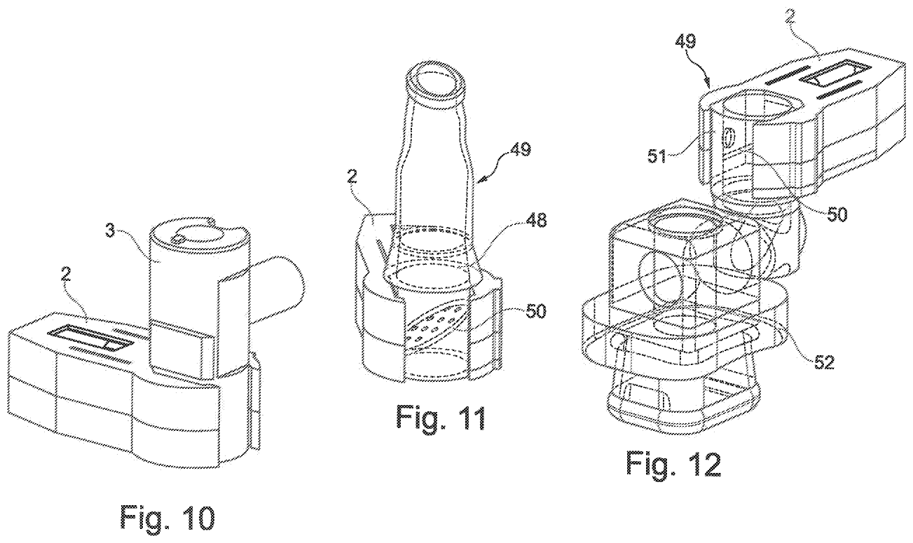

FIG. 10 a measuring adapter on an endoscope head;

FIG. 11 a measuring adapter on a breathing tube of a spirometer;

FIG. 12 a measuring adapter on a bite block;

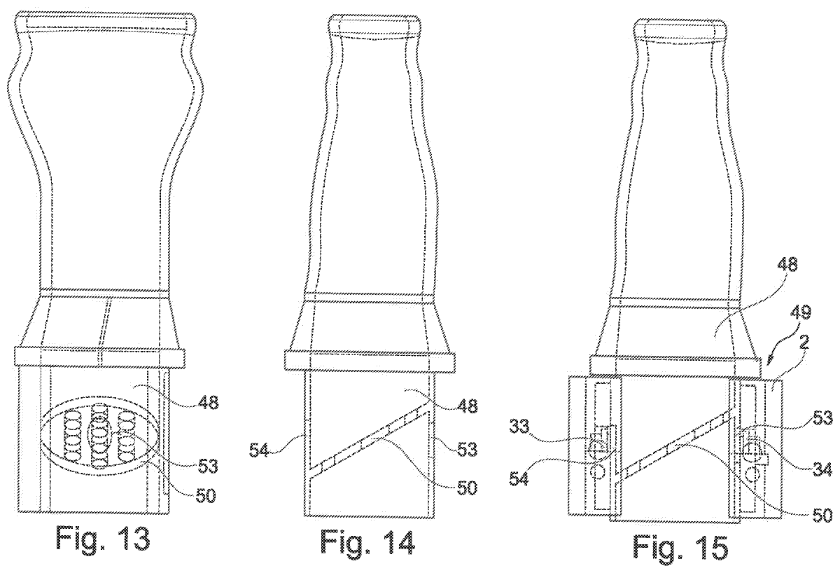

FIG. 13 a first view of a breathing tube of a spirometer;

FIG. 14 a second view of the breathing tube of a spirometer of FIG. 13; and

FIG. 15 the breathing tube of a spirometer according to FIGS. 13 and 14 with attached measuring adapter.

FIG. 1 shows a medical apparatus 1 with a measuring adapter 2, which is connected to an endoscope head 3. The endoscope head 3 carries an endoscope tube 4. The endoscope head 3 is connected to a ventilation unit 5 via ventilation tubes 6, 7 for ventilation or jet ventilation. The measuring adapter 2 is connected to the ventilation unit 5 via a signal-transmitting channel 8. The signal transmission can be wired and/or contactless, for example via Bluetooth. In this design example, an instrument 9 is arranged in the endoscope tube 4.

FIG. 2 shows a medical apparatus 10 with a measuring adapter 11 according to the state of the art (DE 10 2020 110 840 A1). According to the invention, such a measuring adapter 11 is refined. The measuring adapter 11 according to the state of the art is configured as a U-shaped clamp. The endoscope head 12 according to the state of the art has a longitudinal channel 13. The endoscope tube 14 is only partially shown. It can be detachably coupled to the endoscope head 12 via corresponding adapters 15, 16. The measuring adapter 11 can be detachably coupled to the adapter head 13 by inserting it laterally in the direction of arrow P1 onto the essentially cylindrical endoscope head 12. It can be removed again in the opposite direction. No tools are required to attach and remove it. In addition to the actual longitudinal channel 13, which serves as a working channel, the endoscope head 12 has connections for ventilation. These connections are provided when the endoscope head 12 is used during bronchoscopy. A first connection 17 is used for jet ventilation. A second connection 18 is provided for conventional ventilation at lower pressures. The connections 17, 18 can be used alternatingly.

In addition, the endoscope head 12 includes measuring channels 19, 20, which are arranged diametrically. They are located near the distal end of the endoscope head so that they can be brought into overlap with measuring channels 21, 22 in the adapter sleeves 15, 16. The measuring channels 19, 20 feed into the longitudinal channel 13 and have each an inner connection opening 23 and an outer connection opening 24. The outer connection opening 24 is located in a lateral adapter connection area 25. In contrast to the remaining outer contour of the essentially cylindrical endoscope head 12, the adapter connection area 25 is not rounded but flattened. Since the corresponding connection openings 24 are diametrically opposite each other on the outside, the corresponding adapter connection areas 25, 26 are also arranged opposite each other. The measuring adapter 11 can be pushed in the direction of arrow P1 via the flattened adapter connection areas 25, 26. The measuring adapter 11 includes a back 26 and two arms 27, 28, which are connected to the back 26. The arms 27, 28 are resiliently configured to come into contact with the adapter connection areas 25 with their respective contact surfaces 29, 30 formed on the inside of the arms 27, 28 and to establish a fluid-conducting, in particular gas-conducting connection to the outer connection openings 24 of the measuring channels 19, 20 in the endoscope head 12. The arms 27, 28 have a mutual spacing which is matched to the spacing of the adapter connection areas 25.

The measuring channels 19, 20 from the endoscope head 12 are continued by further measuring channels 31, 32 in the measuring adapter 11. Running in longitudinal direction of the arms 27, 28, there is a respective measuring channel 31, 32. The two measuring channels 31, 32 have each connection openings 33, 34 in the contact surfaces 29, 30 facing each other. The measuring

channels 31, 32 in the measuring adapter 11 feed each into connection pieces 35, 36, to each of which a hose can be connected in order to tap and evaluate the pressure at the measuring adapter 11.

The above remarks on the state of the art with regard to the arrangement and function of the measuring channels with regard to the fastening possibility of the measuring adapter 11 and with regard to the configuration of the endoscope head 12 also apply to the endoscope head 3 of FIGS. 1 and 3 and to the measuring adapters 2 in the figures.

The refinement of such a measuring adapter according to the invention is explained below with reference to FIGS. 3 to 9.

FIG. 3 shows the endoscope head 3 with an endoscope tube 4 and the attached measuring adapter 2 in a greatly shortened form. The measuring adapter 2 is also designed here as a U-shaped clamp, with arms 27, 28 embracing the endoscope head 3. The arms 27, 28 are connected to each other via a back 26. Two handles 37, 38 are connected to the back 26. The two handles 37, 38 are located on the side of the back 26 facing away from the arms 27, 28. They are used for handling the measuring adapter 2. A first sensor 39 is located between the handles 37, 38 in the area of the back 26. The sensor 39 is located on the endoscope head 3 at level with an information carrier 40. The information carrier 40 contains information about the design of the endoscope head 3 (FIG. 4). The sensor 39 reads this information and converts it into a signal that can be transmitted to the ventilation unit 5 via channel 8 (FIG. 1). There it can be displayed on a display unit 41. The ventilation unit 5 has a memory unit 42 and an evaluation unit 43. The signals from the first transmitter 39 are evaluated in the evaluation unit 43. Correction factors or correction functions for determining correction factors for ventilation parameters of certain designs of endoscope heads 3 and/or endoscope tubes 4 are stored in the memory unit 42. They are made available to the evaluation unit 43. The determined correction factor is assigned to the identified endoscope head 3. A control unit 44 is designed to adapt the ventilation parameters of the ventilation unit 5 to the detected endoscope head 3 either automatically or after acknowledgement by an operator of the medical apparatus 1 using the correction factor.

It is clear from the illustration in FIG. 4 that the measurement of the first sensor 39 is contactless. The information of the information carrier 40 is in particular optical, e.g. a barcode.

Furthermore, the sectional view of FIG. 4 shows second sensors 45 in an almost diametrical arrangement. The sensors 45 are used to detect whether an endoscope tube 4 is arranged inside the endoscope head 3. In a manner not shown in detail, openings can be provided in the area of the endoscope head 3 for this detection, so that the sensors 45 can also optically ascertain the presence of an endoscope tube.

As FIG. 2 shows, different endoscope tubes 14 can be connected to the endoscope head 12 via corresponding adapters 15, 16. The same applies to the endoscope head 2 according to the invention. The second sensors 45 are used to determine the diameter of the endoscope tube 3.

The measuring clamp can be attached not only directly to the endoscope head 3, but also to the endoscope tube 4. The exemplary embodiment of FIGS. 5 and 6 shows this configuration of the invention. The endoscope head is not illustrated because the measuring adapter 2 is now attached directly to the endoscope tube 4. Also in this case, the arms 27, 28 embrace the endoscope tube 4 to be determined. Information about the endoscope tube 4 can be read out via the first sensor 39 in the same manner as in conjunction with the endoscope head 3. In addition, the second sensors 45 can be used to determine the presence of the endoscope tube 4 and preferably also its diameter. The sensors 45 operate optically in particular. It may involve a sensor arrangement in the form of a light barrier, with this sensor arrangement including a signal source 46 in the form of a light source. The light transmission in the area of the endoscope tube 4 is measured. The scattering of the light can be used to determine whether an endoscope tube 4 is present or how large the diameter of the endoscope tube 4 is. The invention is not limited to the arrangement of a single signal source 46 or a single second sensor 45. Several such sensors 45 can also be arranged dispersed about the circumference in order to enable a more precise determination of the diameter of the endoscope tube 4.

The exemplary embodiment of FIGS. 7 and 8 differs from the one of FIGS. 5 and 6 in that the instrument 9 mentioned in FIG. 1 is inserted into the channel of the endoscope tube 4. The measuring adapter 2 now has the additional function of determining whether an instrument 9 is inserted into the endoscope tube 4. When the type of endoscope tube 4 is known and therefore its light transmission capability, deviations from the previously known values can be used to determine whether an instrument 9 has been inserted into the endoscope tube 4. In the purely schematic illustration of FIGS. 7 and 8, a light path between a signal source 46 and the sensor 45 would be completely interrupted. This is a reliable signal that an instrument 9 is located in the endoscope tube 4. The correction factor can be determined accordingly and ventilation parameters can be adjusted.

The exemplary embodiment in FIG. 9 shows in addition the measuring adapter 2 on an endoscope tube 4, with a display unit 47 directly illustrating whether and which endoscope tube 4, referred to here as “tube B”, has been detected by the second sensor 45 and/or the first sensor 39. The measuring adapter 2 includes therein miniaturized electronics which, in particular in combination with optical sensors, infrared diodes, pressure sensors and one or more microcontrollers, make it possible to display the desired information to an operator as a stand-alone device, in particular the correction factors calculated in real time. An evaluation unit is located in the measuring adapter for this purpose. Via the display on the measuring clamp, the operator is immediately informed of the correction factor and, preferably, which endoscope head is connected, whether an instrument is inserted and which endoscope tube is currently connected.

The medical apparatus according to the invention relates in particular to endoscope heads and endoscope tubes for single use. In particular, these components are made of plastic. The measuring adapter is reusable.

FIG. 10 shows the application of the measuring adapter 2 on the endoscope head 3, while FIG. 12 shows as alternative application the measuring adapter 2 on a breathing tube 48. Together with the measuring adapter 2, the breathing tube 48 forms a spirometer 49, or also only its measuring device. It involves a differential pressure flowmeter in which the flow velocity is determined via pressure differences in the breathing tube 48 as a perforated wall 50 is passed through. The perforated wall 50 is arranged diagonally in the breathing tube 48. The perforated wall 50 is shaped like a sieve. A measuring channel 31 is arranged anteriorly of the perforated wall 50 in the direction of flow and a further measuring channel 32 is arranged posteriorly of the perforated wall 50 in the direction of flow (FIG. 15). The pressure differences are ascertained via the measuring channels 31, 32. The pressure difference can be used to deduce the flow velocity and thus the breathing volumes. The parameters for ventilation associated with the respective breathing tube are recognized by the measuring adapter and used to determine correction factors. When the measuring adapter 2 recognizes that in combination with the measuring adapter 2 a spirometer 49 is formed, the differential pressure is preferably automatically evaluated in an evaluation unit in order to determine the breathing volumes, i.e. switched to the spirometer function.

FIG. 12 shows a breathing tube 51 of a different design on a bite block 52. The connection to the bite block 52 can be detachable. The functional principle is the same as in FIG. 11. It involves a differential pressure flowmeter in which a pressure is measured anteriorly and posteriorly of a perforated wall by means of the measuring adapter 2 in order to determine a pressure difference and thus the breathing volumes.

FIGS. 13 to 15 refer to the exemplary embodiment of FIG. 11. The breathing tube 48 is shown in FIGS. 13 and 14 in two different side views, so that the position of the perforated wall 50 in the breathing tube 48 becomes clear. In addition, the diametrical arrangement of the measuring channels 53, 54 in the breathing tube 48 is depicted. The position of the measuring channels 53, 54 is matched to the measuring adapter 2, which is connected in FIG. 15. The measuring adapter 2 includes the measuring channels 31, 21 shown in FIG. 2 with the terminal openings 33, 34, which in this case are brought into overlap with the measuring channels 53, 54.

### Reference Signs

1-medical apparatus
2-measuring adapter
3-endoscope head
4-endoscope tube
ventilation unit
6-ventilation hose
7-ventilation hose
8-channel for data transmission
9-instrument
medical apparatus
11-measuring adapter
12-endoscope head
13-longitudinal channel from 12
14-endoscope tube
adapter
16-adapter
17-connection to 13
18-connection to 13
19-measuring channel
measuring channel
21-measuring channel in 16
22-measuring channel in 15
23-inner connection opening of 19
24-outer connection opening of 19
adapter connection area on 2
26-back
27-arm
28-arm
29-contact surface
30-contact surface
31-measuring channel in 27
32-measuring channel in 28
33-opening of 31
34-opening
35-connection piece
36-connection piece
37-handle
38-handle
39-first sensor
40-information carrier
41-display unit of 5
42-memory unit of 5
43-evaluation unit of 5
44-control unit of 5
45-second sensor
46-signal source
47-display unit
48-breathing tube
49-spirometer
50-perforated wall
51-breathing tube
52-bite block
53-measuring channel
54-measuring channel
P1-arrow

1-18. (canceled)

19. A medical apparatus, comprising:
a measuring adapter designed for detachable connection to different respiratory gas pathway devices which have ventilation parameters;
a first sensor arranged on the measuring adapter and designed to measure a measured variable of the different respiratory gas pathway devices; and
an evaluation unit designed to assign correction factors to the ventilation parameters based on measured values of the first sensor.

20. The medical apparatus of claim 19, wherein the measuring adapter is designed for detachable connection to the different respiratory gas pathway devices which are selected from the group consisting of endoscope head, endoscope tube, intubation tube, breathing hose, breathing tube of a spirometer, and bite block.

21. The medical apparatus of claim 19, wherein the different respiratory gas pathway devices include an information carrier which are readable by the first sensor.

22. The medical apparatus of claim 19, further comprising a memory unit designed to store a correlation of measurement data to the correction factors of the ventilation parameters for the different respiratory gas pathway devices in order to make the correlation of the measurement data to the correction factors available to the evaluation unit.

23. The medical apparatus of claim 19, further comprising a ventilation unit connected to the measuring adapter in a data-transmitting manner and including a control unit designed to automatically adjust the ventilation parameters by taking into account the correction factors.

24. The medical apparatus of claim 23, wherein at least one of the ventilation unit and the measuring adapter includes a display unit designed to display a corresponding one of the correction factors.

25. The medical apparatus of claim 20, wherein the endoscope head is designed for coupling to endoscope tubes of different diameters, the medical apparatus further comprising a second sensor provided on the measuring adapter to ascertain the diameter of a connected one of the endoscope tubes.

26. The medical apparatus of claim 20, wherein the measuring adapter includes a sensor designed to ascertain a presence of an instrument in the endoscope tube or in the intubation tube.

27. The medical apparatus of claim 26, wherein the sensor of the measuring adapter measures a measured variable, with the endoscope tube or the incubation tube including a permeable region for the measured variable of the sensor of the measuring adapter to ascertain the presence of the instrument in the connected one of the endoscope tubes or the intubation tube.

28. The medical apparatus of claim 27, wherein the permeable region of the endoscope tube or of the intubation tube is translucent.

29. The medical apparatus of claim 28, wherein the translucent region extends over an entire circumference of the endoscope tube or the intubation tube.

30. The medical apparatus of claim 29, further comprising a signal source arranged on the measuring adapter for generating the measured variable of the sensor of the measuring adapter.

31. The medical apparatus of claim 23, wherein a data transmission between the measuring adapter and the ventilation unit is wireless.

32. The medical apparatus of claim 20, wherein the endoscope head includes a measuring channel which has an inner connection opening connected to a longitudinal channel in the endoscope head, and an outer connection opening arranged on an outside of the endoscope head, with the outer connection opening being connectable to a measuring channel in the measuring adapter.

33. The medical apparatus of claim 19, wherein the measuring adapter is configured as a U-shaped clamp with a back and with two arms which are connected to the back.

34. The medical apparatus of claim 33, wherein the first sensor is arranged in the back.

35. The medical apparatus of claim 33, wherein the endoscope head is designed for coupling to endoscope tubes of different diameters, the medical apparatus further comprising a second sensor arranged in or on the arms of the measuring adapter to ascertain the diameter of a connected one of the endoscope tubes.

36. The medical apparatus of claim 19, wherein the measuring adapter comprises an evaluation unit to determine a correction factor in the measuring adapter, and a display unit to display the correction factor on the measuring adapter.
